(19) United States
(12) Patent Application Publication
(10) Pub. No.: US 2026/0063650 A1
(43) Pub. Date: Mar. 5, 2026

(54) METHOD FOR EXCLUDING FACTOR XIA INHIBITORS IN PATIENT SAMPLES

(71) Applicant: Siemens Healthcare Diagnostics Products GmbH, Marburg (DE)

(72) Inventors: Christa DUWE, Gladenbach (DE); Norbert Zander, Marburg (DE); Katharina Naumann, Marburg (DE)

(21) Appl. No.: 19/304,817

(22) Filed: Aug. 20, 2025

(30) Foreign Application Priority Data
Aug. 27, 2024 (EP) 24196609.2

(51) Int. Cl. G01N 33/86

(52) U.S. Cl. CPC G01N 33/86

(57) ABSTRACT

The present disclosure is in the field of coagulation diagnostics and relates to a method for excluding anticoagulants from the group of factor XIa inhibitors in a patient sample.

### CROSS-REFERENCE TO RELATED PATENT APPLICATIONS

This patent application claims foreign priority under 37 U.S.C. § 119 to European Patent Application No. 24196609 filed 27 Aug. 2024, the content of which is hereby incorporated by reference in its entirety.

### FIELD

The present disclosure is in the field of coagulation diagnostics and relates to a method for excluding anticoagulants from the group of factor XIa inhibitors in a patient sample.

### BACKGROUND

The role of the blood coagulation system (hemostasis) is that of, firstly, ensuring a continuous flow of blood and, secondly, bringing about wound closure in a rapid and localized manner in the event of an injury in order to minimize blood loss. A cascade of enzymatic processes, with each enzyme activating a further enzyme through proteolytic processes, ends in fibrin formation, which contributes to the formation of wound-closing blood clots. Examples of such procoagulatory enzymes are thrombin (factor IIa), factor Xa and factor XIa. Thrombin as well as factor Xa and factor XIa are serine proteases which normally occur in blood in the form of inactive proenzymes and are only activated in connection with the activation of coagulation.

Factor XI is a component of the intrinsic blood coagulation system and is activated by activated factor XII or by thrombin and it itself activates factor IX, which then lastly influences thrombin formation via the activation of factor X.

In anticoagulation therapy, there is increasing use of novel direct coagulation factor inhibitors, in particular direct thrombin and factor Xa inhibitors and, most recently, also factor XI inhibitors. These novel anticoagulants have the potential to replace the hitherto used indirect anticoagulants, especially heparin and its derivatives, which only exhibit their anticoagulant effect in cooperation with cofactors such as antithrombin or heparin cofactor II. For the control of therapy and for the dosing of drugs, it is therefore important to know the amount of the therapeutic inhibitor. It is therefore necessary to have diagnostic methods that allow the determination of the presence, activity or plasma concentrations of therapeutic coagulation factor inhibitors.

Known in the prior art are various test methods for quantitatively detecting direct inhibitors of proteolytically active coagulation factors, such as inhibitors of thrombin, factor Xa or factor XIa, in human plasma samples.

Inhibitors of proteolytically active coagulation factors are usually determined with the aid of chromogenic or coagulometric test methods. In the case of chromogenic methods, the patient sample suspected of containing, for example, a thrombin or factor Xa inhibitor is mixed with a defined amount of the corresponding activated coagulation factor present in excess and with a chromogenic substrate for the activated coagulation factor, and the remaining activity of the coagulation factor in the reaction is measured photometrically. In the case of established chromogenic tests, which are also commercially available, use is made in particular of the chromophores para-nitroaniline (pNA) and 5-amino-2-nitrobenzoic acid (ANBA), which have an absorption maximum at 405 nm. The yellow color formed is generally determined photometrically. The higher the concentration of the therapeutic inhibitor in the patient sample, the greater the inhibition of the activity of the coagulation factor added and the less substrate that is cleaved. When determining inhibitors, the color concentration in the test is inversely proportional to the inhibitor concentration in the sample. For example, EP-B1-0034320 or EP-A2-0004271 describes such thrombin- or factor Xa-based chromogenic test methods. With the aid of these tests, therapeutic inhibitors that inhibit the activity of blood coagulation factors can be quantitatively determined in patient samples. WO-A1-2012069139 describes a method for determining factor Xa inhibitors and WO-A1-2012175183 describes a method for determining thrombin inhibitors in serum and urine samples.

As the use of a wide variety of coagulation factor inhibitors for the treatment or prophylaxis of thrombotic diseases increases, so is there a growing need to provide rapid and simple diagnostic methods that make it possible to determine whether a patient, for example a patient admitted to a hospital in an unconscious state without any information available about their medical history or medication, is being treated with anticoagulants and, if so, what they are. Especially in the case of surgical procedures that must be carried out urgently, knowledge of the blood coagulation status of a patient and any anticoagulant therapy is important for the assessment of the risk of bleeding complications.

In this context, it is desirable to have a simple and rapid test by means of which the presence of an anticoagulant from the group of factor XIa inhibitors in a patient sample can be excluded.

### SUMMARY

In one embodiment, an object of the present invention is to provide a simple method for excluding anticoagulants from the group of factor XIa inhibitors in a patient sample, said method having high sensitivity and specificity for the absence of factor XIa inhibitors.

In one embodiment, the object is achieved by providing, on the basis of a conventional APTT test that is typically available in any clinical laboratory, a method in which the patient sample and a normal plasma sample are first mixed with a factor XI-deficient plasma and with at least one neutralizing substance which neutralizes an anticoagulant other than an anticoagulant from the group of factor XIa inhibitors and the APTT (activated partial thromboplastin time) is determined in the two samples. The thus determined coagulation times of the two samples are related to each other by forming a quotient, and the quotient is compared with a predetermined reference value, it being possible to exclude the presence of a factor XIa inhibitor in the patient sample if the quotient is below the predetermined reference value.

### BRIEF DESCRIPTION OF THE DRAWINGS

FIG. 1 shows a graphical representation of the quotients APTT XI_{Sample}/APTT XI_{Normal} formed according to the invention for different samples and calculated from the inventively determined APTT coagulation times of a sample and a normal plasma sample, compared to the quotients APTT_{Sample}/APTT_{Normal} calculated from the conventionally determined APTT coagulation times of a sample and a normal plasma sample. For samples having a quotient APTT XI_{Sample}/APTT XI_{Normal} below the cut-off value of 1.21, the presence of ≥1 μM asundexian (therapeutic factor XIa inhibitor) can be excluded.

### DETAILED DESCRIPTION

In one embodiment, a method is provided for excluding anticoagulants from the group of factor XIa inhibitors in a patient sample, the method comprising the steps of
a) preparing a first reaction mixture containing the patient sample and a second reaction mixture containing a normal plasma sample by mixing each of the two samples with
b) a factor XI-deficient plasma, and
c) at least one neutralizing substance which neutralizes an anticoagulant other than an anticoagulant from the group of factor XIa inhibitors, and
d) a coagulation activator of the intrinsic blood coagulation system;
e) measuring the coagulation time in the first reaction mixture (APTT XI_{Sample}) and the coagulation time in the second reaction mixture (APTT XI_{Normal});
f) forming the quotient APTT XI_{Sample}/APTT XI_{Normal} from the two coagulation times;
g) comparing the quotient with a predetermined reference value; and
h) excluding anticoagulants from the group of factor XIa inhibitors in the patient sample if the quotient APTT XI_{Sample}/APTT XI_{Normal} is below the predetermined reference value.

The term “anticoagulants from the group of factor XIa inhibitors” refers to a therapeutically active substance which does not naturally occur in the human body and which specifically reduces the proteolytic activity of coagulation factor XIa, typically by direct binding to factor XIa (or XI). The term “anticoagulants from the group of factor XIa inhibitors” explicitly excludes anticoagulant proteins or protein complexes which naturally occur in the human body, such as antithrombin or heparin cofactor II, and which can directly or indirectly inhibit the proteolytic activity of coagulation factor XIa.

Examples of currently known anticoagulants from the group of factor XIa inhibitors include asundexian, milvexian and abelacimab, and also osocimab, gruticibart (xisomab), REGN9933, MK-2060, ONO-7684 or ONO-5450598.

A “patient sample” is to be understood to mean a plasma sample, for example a citrate plasma sample, from an individual.

A “normal plasma sample” is to be understood to mean a plasma sample from an apparently healthy blood donor or a sample of a mixture of plasmas from multiple, for example 20, apparently healthy blood donors, i.e., a normal plasma pool. In a normal plasma, the values of all coagulation factors are within the normal range.

The term “factor XI-deficient plasma” refers to a plasma of human or other animal origin from which factor XI has been completely or at least virtually removed, for example by immunoadsorption, while all other coagulation factors are present in normal concentrations. Typically, plasmas from multiple apparently healthy blood donors (human or animal) are pooled to form a normal plasma pool, followed by removal or substantial removal of factor XI from said normal plasma pool by adsorption, for example using factor XI-specific monoclonal and/or polyclonal antibodies, thereby yielding a factor XI-deficient plasma.

According to the invention, the patient sample and the normal plasma sample are mixed with factor XI-deficient plasma by adding preferably two parts by volume of factor XI-deficient plasma to one part by volume of patient sample or normal plasma sample. Diluting the patient sample with factor XI-deficient plasma brings about compensation for any coagulation factor deficiencies in the patient sample, meaning that they will have no influence on the later coagulation reaction.

Also added to the patient sample and the normal plasma sample is at least one neutralizing substance which neutralizes an anticoagulant other than an anticoagulant from the group of factor XIa inhibitors. The purpose of this is to eliminate the coagulation time-extending effect of any other anticoagulants that might be present in the patient sample, meaning that they will have no influence on the later coagulation reaction. The anticoagulants that should preferably be neutralized can vary depending on the time and place, since specific anticoagulants can vary in popularity over the years or in the health systems of different countries. Multiple neutralizing substances which neutralize an anticoagulant other than, or various anticoagulants other than, an anticoagulant from the group of factor XIa inhibitors may also be added to the patient sample and the normal plasma sample.

Preferably, the at least one anticoagulant-neutralizing substance is a substance which neutralizes an anticoagulant from the group of thrombin and FXa inhibitors. A suitable thrombin inhibitor-neutralizing substance is, for example, idarucizumab; suitable FXa inhibitor-neutralizing substances are, for example, andexanet alfa and ciraparantag.

Particularly preferably, the at least one anticoagulant-neutralizing substance is a substance which neutralizes heparin. A suitable heparin-neutralizing substance is, for example, protamine sulfate.

To prepare a first reaction mixture, a coagulation activator of the intrinsic blood coagulation system is then added to the patient sample mixed with a factor XI-deficient plasma and with at least one neutralizing substance which neutralizes an anticoagulant other than an anticoagulant from the group of factor XIa inhibitors. Analogously, to prepare a second reaction mixture, the same coagulation activator of the intrinsic blood coagulation system is added to the normal plasma sample mixed with a factor XI-deficient plasma and with at least one neutralizing substance which neutralizes an anticoagulant other than an anticoagulant from the group of factor XIa inhibitors.

A coagulation activator of the intrinsic blood coagulation system typically consists of a combination of phospholipids (so-called partial thromboplastins), calcium ions and a surface-active substance, such as kaolin or ellagic acid or silica, etc. Adding a coagulation activator of the intrinsic blood coagulation system to a blood or plasma sample initiates blood coagulation in the reaction mixture.

What is then measured in the two reaction mixtures is the time (in seconds) until clot formation occurs, i.e., the coagulation time or, more precisely, the activated partial thromboplastin time (APTT). The coagulation time may be determined by manual or automatic methods. In the case of automatic determination, measuring a mechanical or optical property of the reaction mixture, for example the viscosity or turbidity, is very widespread. In all cases of automatic measurement, a property of the reaction mixture is continuously measured and, with the aid of conventional evaluation methods, the time-dependent change in the property can be used to determine the coagulation time as the end point.

According to the invention, what are measured are the coagulation time in the first reaction mixture containing the patient sample (APTT XI_{Sample}) and the coagulation time in the second reaction mixture containing the normal plasma sample (APTT XI_{Normal}). The two coagulation times thus determined are then used to form the quotient APTT XI_{Sample}/APTT XI_{Normal}, and the quotient formed is compared with a predetermined reference value.

The reference quotient is usually determined in advance, typically by determining the coagulation time for a statistically sufficient number of samples from patients known to be therapied with an anticoagulant from the group of factor XIa inhibitors and from apparently healthy normal donors who are untreated and optionally from patients known to be therapied with an anticoagulant other than an anticoagulant from the group of factor XIa inhibitors (e.g., with heparin or a direct thrombin or factor Xa inhibitor) by means of the modified APTT test according to the invention (addition of FXI-deficient plasma and an anticoagulant-neutralizing substance), forming quotients of the coagulation times of the factor XIa inhibitor-positive samples and the factor XIa inhibitor-negative samples, and relating them to the coagulation time(s) of normal plasma samples. From the set of quotients formed, a reference value (reference quotient, limit value, cut-off value) allowing extremely reliable differentiation between factor XIa inhibitor-negative and factor XIa inhibitor-positive patient samples can then be determined.

It has been found that the presence of an anticoagulant from the group of factor XIa inhibitors in the patient sample can be excluded if the quotient APTT XI_{Sample}/APTT XI_{Normal} is below the predetermined reference value.

In another embodiment, a test kit is provided for use in a method for excluding anticoagulants from the group of factor XIa inhibitors in a patient sample, the test kit containing:
i) a factor XI-deficient plasma, and
j) at least one neutralizing substance which neutralizes an anticoagulant other than an anticoagulant from the group of factor XIa inhibitors.

The factor XI-deficient plasma and the at least one neutralizing substance (both as described above) may be each provided as a separate reagent, in liquid form or as a resuspendable lyophilisate. Alternatively, the test kit may contain a reagent containing a mixture of the factor XI-deficient plasma and the at least one neutralizing substance.

The reagent containing the at least one neutralizing substance which neutralizes an anticoagulant other than an anticoagulant from the group of factor XIa inhibitors, irrespective of whether it is a separate reagent or a reagent additionally containing the factor XI-deficient plasma or a reagent additionally containing a coagulation activator of the intrinsic blood coagulation system (see below), preferably contains a neutralizing substance which neutralizes an anticoagulant from the group of thrombin and FXa inhibitors, such as the thrombin inhibitor-neutralizing substance idarucizumab and/or at least one of the FXa inhibitor-neutralizing substances andexanet alfa and ciraparantag. Particularly preferably, the reagent contains the heparin-neutralizing substance protamine sulfate.

Preferably, a test kit according to the invention additionally contains a coagulation activator of the intrinsic blood coagulation system. The coagulation activator is preferably provided as a separate reagent, in liquid form or as a resuspendable lyophilisate, and preferably contains a combination of phospholipids and a contact activator, preferably a combination of phospholipids and a contact activator from the group consisting of ellagic acid, kaolin and silica. The coagulation activator reagent may also contain calcium chloride. Alternatively, the calcium chloride may be present in the test kit as a separate reagent.

In yet another embodiment, the test kit may contain a reagent containing a mixture of the coagulation activator of the intrinsic blood coagulation system and the at least one neutralizing substance.

In another embodiment, the use of a test kit according to the invention in a method for excluding anticoagulants from the group of factor XIa inhibitors in a patient sample is provided, preferably in a method as described above.

The following examples serve for illustration and are not to be understood as limiting.

### EXAMPLES

### Example 1: Determination of the Conventional APTT And of the Modified APTT According to the Invention (APTT XI) in Samples Differing in Blood Coagulation Status

For the activation of the intrinsic blood coagulation system, an APTT reagent (Dade Actin FS Activated PTT Reagent, Siemens Healthineers) containing ellagic acid and phospholipids was used. To determine the conventional APTT, 50 μL of an (undiluted) sample were mixed with 50 μL of the APTT reagent. After three minutes of incubation at 37° C., 50 μL of a 25 mM calcium chloride solution were added to the reaction mixture, and the APTT coagulation time in seconds was determined on an automatic analyzer.

To carry out the method according to the invention, said APTT reagent was modified such that it additionally contained the heparin-neutralizing substance protamine sulfate (2 μg/mL) in addition to the ellagic acid and the phospholipids. Furthermore, before adding the modified APTT reagent, the samples were mixed with factor XI-deficient plasma by mixing one part by volume of sample with 2 parts by volume of factor XI-deficient plasma. To determine the modified APTT XI according to the invention, 50 μL of a sample mixed with factor XI-deficient plasma were mixed with 50 μL of the modified APTT reagent. After three minutes of incubation at 37° C., 50 μL of a 25 mM calcium chloride solution were added to the reaction mixture, and the APTT XI coagulation time in seconds was determined on an automatic analyzer.

Plasma samples having different coagulation disorders and known to have an APTT coagulation time longer than normal were tested, for example samples having coagulation factor deficiencies, plasma samples containing various anticoagulants or containing other interfering factors (e.g., lupus anticoagulant) (see Table 1).

In particular, the following sample types were tested:

| Sample type | Description |
|---|---|
| Normal plasma pool | Plasma pool of >20 healthy donors; |
| Asundexian | Normal plasma spiked with |
| | different amounts of asundexian |
| | (factor XIa inhibitor); |
| Rivaroxaban control | Normal plasma spiked with |
| | rivaroxaban (factor Xa inhibitor); |
| Factor VIII-, IX-, XI- | Factor VIII-, IX-, XI- or XII- |
| or XII-deficient | depleted plasmas (<1% factor |
| plasma | content) or mixtures of a depleted |
| | plasma with normal plasma; |
| Low-molecular-weight | Normal plasma spiked with low- |
| heparin control | molecular-weight heparin; |
| Heparin control | Normal plasma spiked with |
| | unfractionated heparin; |
| Control with reduced | Plasma pool with reduced activity |
| factor content | of intrinsic coagulation factors |
| | (factor VIII, IX, XI or XII); |
| Lupus anticoagulant | Plasmas from individual donors, |
| control | lupus anticoagulant-positive in a |
| | test system based on the dilute |
| | Russell's viper venom (DRVVT) test. |

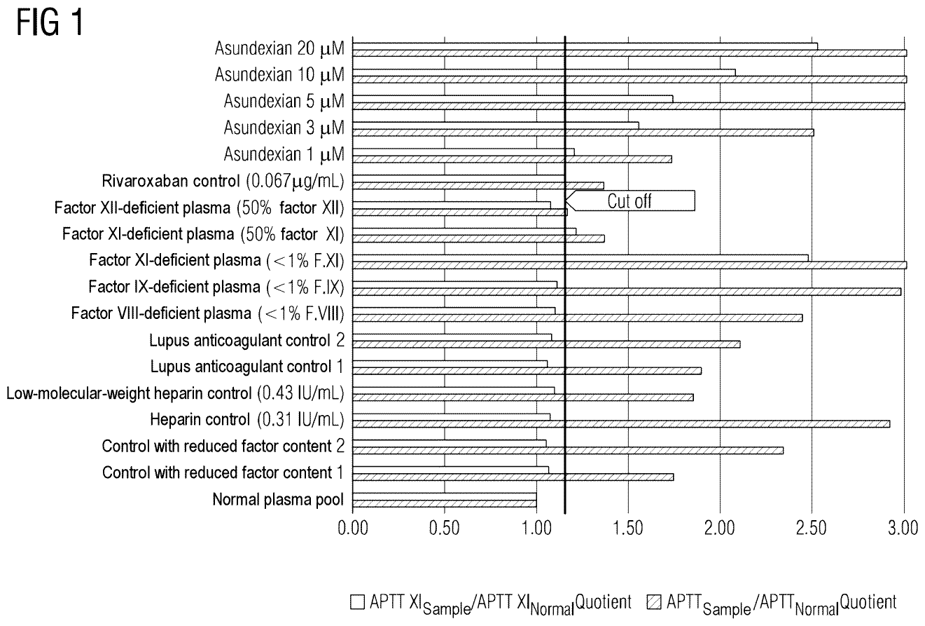

Determined for each sample were the conventional APTT (APTT) and the modified APTT (APTT XI) according to the invention in seconds, and calculated for each sample were 1.) the quotient of the APTT determined for the sample and the APTT of a normal plasma pool (APTT_{Sample}/APTT_{Normal}) and 2.) the quotient of the APTT XI determined for the sample and the APTT XI of the normal plasma pool (APTT XI_{Sample}/APTT XI_{Normal}) (see Table 1). The results are also shown graphically in FIG. 1.

TABLE 1

| | | | | APTT |
|---|---|---|---|---|
| | | | | XI_{Sample}/ |
| | APTT | APTT | APTT_{Sample}/ | APTT |
| Sample | [s] | XI [s] | APTT_{Normal} | XI_{Normal} |
| Normal plasma | 28.19 | 44.7 | 1.00 | 1.00 |
| Asundexian, 1 μM | 48.95 | 53.89 | 1.74 | 1.21 |
| Asundexian, 3 μM | 70.74 | 69.52 | 2.51 | 1.56 |
| Asundexian, 5 μM | 84.67 | 77.89 | 3.00 | 1.74 |
| Asundexian, 10 μM | 106.7 | 93.09 | 3.79 | 2.08 |
| Asundexian, 20 μM | 130.8 | 113.9 | 4.64 | 2.53 |
| Rivaroxaban control, | 38.5 | 51.6 | 1.37 | 1.15 |
| 67 ng/mL | | | | |
| Factor VIII-deficient | 68.99 | 49.22 | 2.45 | 1.10 |
| plasma (<1% F.VIII) | | | | |
| Factor IX-deficient | 84.1 | 49.63 | 2.98 | 1.11 |
| plasma (<1% F.IX) | | | | |
| Factor XI-deficient | 119.2 | 110.8 | 4.23 | 2.48 |
| plasma (<1% F.XI) | | | | |
| Factor XI-deficient | 38.6 | 54.3 | 1.37 | 1.21 |
| plasma (<50% F.XI) | | | | |
| Factor XII-deficient | 32.9 | 48.1 | 1.17 | 1.08 |
| plasma (<50% F.XII) | | | | |
| Low-molecular-weight | 82.35 | 47.97 | 1.85 | 1.10 |
| heparin control | | | | |
| (0.43 IU/mL) | | | | |
| Heparin control | 52.26 | 49.01 | 1.90 | 1.07 |
| (0.31 IU/mL) | | | | |
| Lupus anticoagulant | 53.44 | 47.32 | 1.90 | 1.06 |
| control 1 (low-positive) | | | | |
| Lupus anticoagulant | 59.41 | 48.35 | 2.11 | 1.08 |
| control 2 (high-positive) | | | | |
| Control with reduced | 49.23 | 47.61 | 1.75 | 1.05 |
| factor content 1 | | | | |
| Control with reduced | 66.05 | 47 | 2.34 | 1.07 |
| factor content 2 | | | | |

The results show that a quotient APTT XI_{Sample}/APTT XI_{Normal} determined according to the invention of <1.21 in the group of samples tested excludes the presence of ≥1 μM asundexian (therapeutic factor XIa inhibitor) in the sample. Only samples highly deficient in factor XI (≤50%) cannot be reliably excluded; however, since this disease is rare, there is no appreciable effect on the specificity of the method in practice. By contrast, the quotient APTT_{Sample}/APTT_{Normal} calculated from the conventionally determined APTT coagulation times of a sample and a normal plasma sample is not capable of differentiating between factor XIa inhibitor-positive and factor XIa inhibitor-negative samples.

The exclusion of factor XIa inhibitors using the presently determined cut-off value of 1.21 is not expected to be fully selective in large patient populations. The best possible cut-off value must —as is customary in such cases—be determined in a clinical study with a statistically sufficient number of patient samples and with the aid of a receiver operator characteristic analysis, with a balance having to be made between sensitivity and specificity.

1. A method for excluding an anticoagulant from the group of a factor XIa inhibitors in a patient sample, the method comprising the steps of:
a) preparing a first reaction mixture containing the patient sample and a second reaction mixture containing a normal plasma sample by mixing each of the patient sample and the plasma sample with
i) a factor XI-deficient plasma, and
ii) at least one neutralizing substance which neutralizes an anticoagulant other than an anticoagulant from the group of factor XIa inhibitors, and
iii) a coagulation activator of the intrinsic blood coagulation system;
b) measuring a first coagulation time in the first reaction mixture (APTT XI_{Sample}) and a second coagulation time in the second reaction mixture (APTT XI_{Normal});
c) forming a quotient APTT XI_{Sample}/APTT XI_{Normal} from the first and the second coagulation times;
d) comparing the quotient with a predetermined reference value; and
e) excluding anticoagulants from the group of factor XIa inhibitors in the patient sample if the quotient APTT XI_{Sample}/APTT XI_{Normal} is below the predetermined reference value.

2. The method as claimed in claim 1, wherein the at least one neutralizing substance neutralizes an anticoagulant from the group of thrombin inhibitors and FXa inhibitors.

3. The method as claimed in claim 2, wherein the at least one neutralizing substance in respect of an anticoagulant from the group of thrombin inhibitors is idarucizumab and the at least one neutralizing substance in respect of an anticoagulant from the group of FXa inhibitors is andexanet alfa or ciraparantag.

4. The method as claimed in claim 2, wherein the at least one neutralizing substance neutralizes heparin.

5. The method as claimed in claim 4, wherein the at least one heparin-neutralizing substance is protamine sulfate.

6. The method as claimed in claim 1, wherein the factor XIa inhibitor excluded is selected from the group consisting of asundexian, milvexian, abelacimab, osocimab, gruticibart (xisomab), REGN9933, MK-2060, ONO-7684, and ONO-5450598.

7. A test kit for use in a method for excluding anticoagulants from the group of factor XIa inhibitors in a patient sample, the test kit comprising:
a) a factor XI-deficient plasma, and
b) at least one neutralizing substance which neutralizes an anticoagulant other than an anticoagulant from the group of factor XIa inhibitors.

8. The test kit as claimed in claim 7, wherein the at least one neutralizing substance neutralizes an anticoagulant from the group of thrombin inhibitors and FXa inhibitors.

9. The test kit as claimed in claim 8, wherein the at least one neutralizing substance in respect of an anticoagulant from the group of thrombin inhibitors is idarucizumab and the at least one neutralizing substance in respect of an anticoagulant from the group of FXa inhibitors is andexanet alfa or ciraparantag.

10. The test kit as claimed in claim 8, wherein the at least one neutralizing substance neutralizes heparin.

11. The test kit as claimed in claim 10, wherein the at least one neutralizing substance is protamine sulfate.

12. The test kit as claimed in claim 7, further comprising a coagulation activator of the intrinsic blood coagulation system.

13. The test kit as claimed in claim 12, wherein the coagulation activator of the intrinsic blood coagulation system is a combination of phospholipids and a contact activator.

14. The test kit as claimed in claim 13, wherein the coagulation activator of the intrinsic blood coagulation system is a combination of phospholipids and a contact activator selected from the group consisting of ellagic acid, kaolin, and silica.

15. The test kit as claimed in claim 7, further containing calcium chloride.

16. The test kit as claimed in claim 7, wherein the test kit comprises a reagent containing a mixture of the factor XI-deficient plasma and the at least one neutralizing substance.

17. The test kit as claimed in claim 12, wherein the test kit comprises a reagent containing a mixture of the coagulation activator of the intrinsic blood coagulation system and the at least one neutralizing substance.
